(19) United States
(12) Patent Application Publication
(10) Pub. No.: US 2023/0377649 A1
(43) Pub. Date: Nov. 23, 2023

(54) Systems And Methods For Digital Information Decoding And Data Storage In Hybrid Macromolecules

(71) Applicant: ECOLE POLYTECHNIQUE FEDERALE DE LAUSANNE (EPFL), Lausanne (CH)

(72) Inventors: Matteo DAL PERARO, Preverenges (CH); Chan CAO, Ecublens (CH); Aleksandra RADENOVIC, Ecublens (CH); Lucien KRAPP, Villars-Tiercelin (CH); Jean-Francois LUTZ, Strasbourg (FR); Abdelaziz AL OUAHABI, Strasbourg (FR); Niklas Felix KONIG, Berlin (DE)

(21) Appl. No.: 18/027,566

(22) PCT Filed: Sep. 29, 2020
(86) PCT No.: PCT/EP2020/077229

§ 371 Date: Mar. 21, 2023

(51) Int. Cl. G11C 13/02; G16B 50/30; G16B 50/50

(52) U.S. Cl. CPC G11C 13/02; G16B 50/30; G16B 50/50

(57) ABSTRACT

Systems and methods for data storage and readout using hybrid nucleic acid-polymeric molecules are herein disclosed. A molecular data storage medium is presented comprising a header and a footer, each comprising or consisting of at least one unit of a first chemical species; and a sequence-controlled polymeric chain of a second chemical species located between said header and said footer, said polymeric chain encoding for a desired bitstream-format media. A nanopore-based device and methods for decoding digital information from the molecular data storage

### TECHNICAL FIELD

The present disclosure belongs to the field of molecular computing and digital data storage/decoding. In particular, herein provided are systems and methods for data storage and readout using for instance hybrid nucleic acid-polymeric molecules.

### BACKGROUND ART

As digital information continues to accumulate, higher density and long-term storage are necessary. Data storage capability has been a key aspect of the latest technological developments of human-kind and it still is as never before a compelling challenge to face the current and future “big data” explosion. Although the research on semiconductors have dramatically improved the capacity of data storage in silicon devices, this technology cannot meet the exponential growth of demand for digital data production and storage. This issue is expected to keep widening, as data storage density of silicon chips is limited and magnetic tapes used to maintain large-scale permanent archives begin to deteriorate after 20 years.

As DNA has evolved to store genetic information at large scales, it was proposed to be used as alternative support for data storage, as it can provide both higher density and longer-term storage. Precise synthesis of sequence-encoded heteropolymers has recently opened the possibility of storing information at the molecular scale with ultrahigh density and long-term storage persistence. However, writing strategies are still complicated and reading options not always practical.

The two essential requirements for molecular data storage at single-molecule level are writing and sequence-reading mechanisms. For writing purpose, chemists have developed numerous synthetic methods of controlling sequences in chain-growth and step-growth polymerizations to build sequence-defined macromolecules in a straightforward and protecting-group-free way.

Meanwhile, nanopore technique, the next generation of sequencing tool, have been explored to read DNA sequence in a fast way. When a ssDNA passes through the nanopore, its sequence can be characterized by variation of ionic currents caused by different nucleobases. More recently, nanopores have been explored for sensing digitally encoded DNA nanostructures.

Nanopore sensing is an approach that relies on the exploitation of individual binding or interaction events between to-be-analysed molecules and pore-forming macromolecules. Nanopore sensors can be created by placing nanometric-scaled pore peptide structures in an insulating membrane and measuring voltage-driven ionic transport through the pore in the presence of substrate molecules. The identity of a substrate can be ascertained through its peculiar electrical signature, particularly the duration and extent of current block and the variance of current levels. Two of the essential components of sequencing nucleic acids using nanopore sensing are (1) the control of nucleic acid movement through the pore and (2) the discrimination of nucleotides as the nucleic acid polymer is moved through the pore.

Pore-forming proteins are produced by a variety of organisms and are often involved in defense or attack mechanisms. One notable feature is that they are produced as soluble proteins that subsequently oligomerize and convert into a transmembrane pore in the target membrane. The most extensively characterized pore-forming proteins are the bacterial pore-forming toxins (PFTs), which, depending on the secondary structure elements that cross the bilayer, have been classified as α- or β-PFTs.

In the past, to achieve nucleotide discrimination the nucleic acid has been passed through a mutant of hemolysin (WO 2014/100481). This has provided current signatures that have been shown to be sequence dependent. It has also been shown that a large number of nucleotides contribute to the observed current when a hemolysin pore is used, making a direct relationship between observed current and polynucleotide challenging.

While the current range for nucleotide discrimination has been improved through mutation of the hemolysin pore, a sequencing system would have higher performance if the current differences between nucleotides could be improved further. In addition, it has been observed that when the nucleic acids are moved through a pore, some current states show high variance. It has also been shown that some mutant hemolysin pores exhibit higher variance than others. While the variance of these states may contain sequence specific information, it is desirable to produce pores that have low variance to simplify the system.

In another approach, mutant forms of lysenin, as well as analyte characterisation using thereof, has been described (WO 2013/153359). Lysenin (also known as efLI) is a pore-forming toxin purified from the coelomic fluid of the earthworm Eisenia fetida. It specifically binds to sphingomyelin, which inhibits aerolysin-induced hemolysis. In still another approach, mutant forms of the pore-forming Msp monomer, as well as analyte characterisation using thereof, has been described (WO 2012/107778).

Cao C. et al. (Nat. Nanotechnol. 2016 Apr. 25. doi: 10.1038/nnano.2016.66) demonstrated the ability of aerolysin nanopore to resolve at high resolution individual short oligonucleotides that are 2 to 10 bases long without any extra chemicals or modifications, useful for single-molecule analysis of oligonucleotides.

International patent application WO 2017/189914 discloses methods for controlled segregation of blocks of information encoded in the sequence of a biopolymer, such as nucleic acids and polypeptides, with rapid retrieval based on multiply addressing nanostructured data. In some embodiments, sequence controlled polymer memory objects include data-encoded biopolymers of any length or form encapsulated by natural or synthetic polymers and including one or more address tags. The sequence address labels are used to associate or select memory objects for sequencing readout, enabling organization and access of distinct memory objects or subsets of memory objects using Boolean logic. In some embodiments, a memory object is a single-stranded nucleic acid scaffold strand encoding bit stream information that is folded into a nucleic acid nanostructure of arbitrary geometry, including one or more sequence address labels.

International patent application WO 2018/081745 discloses methods, systems and devices for reading data stored in a polymer (e.g., DNA) and for verifying the sequence of a polymer synthesized in situ in a nanopore-based chip, said method comprising providing a resonator having an inductor and a cell, the cell having a nanopore and a polymer that can traverse through the nanopore, the resonator having an AC output voltage frequency response at a probe frequency in response to an AC input voltage at the probe frequency, providing the AC input voltage having at least the probe frequency, and monitoring the AC output voltage at least at the probe frequency, the AC output voltage at the probe frequency being indicative of the data stored in the polymer at the time of monitoring, wherein the polymer includes at least two monomers having different properties causing different resonant frequency responses.

The articles “Translocation of precision polymers through biological nanopores” (M. Boukhet et al., Macromolecular Rapid Communications, 38, 1700680, 2017), “Tuning Polymer-Protein Interactions with Salt (M. Talarimoghari et al., Biophysical Journal, 112, 457a, 2017) and “Translocation of Sequence-controlled Synthetic Polymers through Biological Nanopores” (M. Boukhet et al., Biophysical Journal, 114, 182a, 2018) describe threading but not sequencing of macromolecular analytes in non-modified hemolysin and aerolysin nanopores.

There is still a need for alternative solutions with regards to molecular systems and methods for encoding, storing and decoding data information which are simple, robust, precise and reliable.

### SUMMARY OF INVENTION

In order to address and overcome at least some of the above-mentioned drawbacks of the prior art solutions, the present inventors developed a brand new tool for encoding and decoding information having improved features and capabilities.

In particular, a first purpose of the present invention is that of providing a novel molecular medium able to encode information, such as in a bitstream-format, which is relatively easy to synthesise, accurate to decipher and gathering high density of information.

A further purpose of the present invention is that of providing a method for encoding and decoding information based on a molecular data storage medium.

Still a further purpose of the present invention is that of providing a decoding system based on nanopore technology that can precisely and reliably decode information stored in a molecular data storage medium.

All those aims have been accomplished with the present invention, as described herein and in the appended claims.

Inspired by recent progresses presented in the previous background section, the present inventors encoded individual binary information through sequence-controlled DNA-polymer hybrid structures and decoded them using solid state or biological nanopores based on engineered pore-forming toxin aerolysin. In non-limiting embodiments detailed later on along the present disclosure, by a rational and synergic development of aerolysin mutants and the design of DNA nucleobases intercalated on sequence-encoded heteropolymers, the translocation speed of the hybrid molecule can be optimized to have a uniquely identifiable level-by-level signal, which delivered digital reading with single-bit resolution without compromising information density.

Using in one embodiment a deep learning strategy to process the current signal, the present inventors demonstrated the ability of engineered aerolysin nanopores to accurately read the information encoded in hybrids DNA-polymer molecules alone and in mixed samples. These findings open promising possibilities to develop writing-reading techniques to process digital data using a biological-inspired platform. In embodiments of the invention, the molecular data storage medium was designed in a binary format, with n-propyl-phosphate representing bit-0 and (2,2-dipropargyl)-propyl-phosphate representing bit-1. Each bit is characterized by peculiar current levels, as well as DNA bases. By using deep learning, the reading accuracy of 1-bit, 2-bit, 3-bit, and 4-bit barcodes were assessed at 98.7%, 96.4%, 95.0% and 76.9%, respectively, thereby demonstrating the ability of nanopores as polymer sequence decoders, opening the venue for further design of polymers specific for a particular reading pore.

In view of the above, according to the present invention there is provided a molecular data storage medium according to claim 1.

Another object of the present invention relates to a method for encoding a bitstream-format information in a molecular data storage medium according to claim 6.

Still another object of the present invention relates to a nanopore-based device for reading data stored in a molecular data storage medium according to claim 8.

Still another object of the present invention relates to a method for decoding a bitstream-format information encoded in the molecular data storage medium according to claim 13.

Further embodiments of the present invention are defined by the appended claims.

The above and other objects, features and advantages of the herein presented subject-matter will become more apparent from a study of the following description with reference to the attached figures showing some preferred aspects of said subject-matter.

### BRIEF DESCRIPTION OF DRAWINGS

FIG. 1: Aerolysin reading of polymers encoding single-bit information. (a) Illustration of single-channel recording setup using an aerolysin pore; the cis and trans chambers are filled with 1.0 M KCl electrolyte buffer and voltage is applied across the pore using two Ag/AgCl electrodes. (b) Schematic structure of two representative polymers: AA00000AA and AA00100AA. (c) Raw current trace of AA00000AA during single-channel recording measurement (top). Magnification of one single event (bottom). (d) Raw current trace of AA00100AA measurement. Magnification of one single event (bottom) showing a multi-level signal: L-1, L-2, L-3, L-4 and L-5. (e) I/I₀ histogram (top) and dwell time distribution (bottom) for L-1, L-2, L-3, L-4 and L-5, respectively. Relative fitted values are reported in each figure. All data were obtained using 1.0 M KCl, 10 mM Tris, and 1.0 mM EDTA at pH=7.4 applying a bias potential of 100 mV.

FIG. 2: Effects of terminal nucleobases on polymer reading. (a) Representative translocation events of the digital macromolecular analyte with different types of nucleotides at the chain termini. (b) Mean dwell time and mean current variation for all polymers with different types of terminal dinucleotides. All data were obtained using 1.0 M KCl, 10 mM Tris, and 1.0 mM EDTA buffer at pH=7.4 applying a bias potential of 100 mV. Each value is an average obtained from at least three separate pore measurements.

FIG. 3: Decoding polymer sequences using aerolysin pores and deep learning. (a) Details for nanopore signal processing and relative deep learning workflow. (b) Characteristic translocation events of polymers containing bit-1 at different positions, i.e., A01000AA, AA00100AA and AA00010AA, and the corresponding confusion matrix results obtained by deep learning. (c) Confusion matrix of 1-, 2-, 3- and 4-bit polymer sequences classification. Columns represent true polymers from the test set, while rows are the polymers that deep learning assigned them to. All data were obtained using 1.0 M KCl, 10 mM Tris, and 1.0 mM EDTA buffer at pH=7.4 applying a bias potential of 100 mV.

FIG. 4: Statistical analysis and assignment of specific polymer's identity and relative concentration in a mixture. Statistical analysis of (a) mean dwell time and (b) current variation of all polymers. (c) Assignment percentage of blind polymer samples #1, #2, #3, and #4 respectively. Assignment percentage of mixture sample #A (d) and mixture sample #B (e). The theoretical accuracy is shown by the columns. All data were obtained using 1.0 M KCl, 10 mM Tris, and 1.0 mM EDTA buffer at pH=7.4 applying a bias potential of 100 mV.

FIG. 5: a) Chemical structure of all experimentally tried subunits; b) general molecular structure of the molecular data storage media (‘0’ is the same for all polymers); c) chemical structure of possible alternative, non-limiting subunits.

FIG. 6: Detection of polymer ‘00000’ and ‘11111’ by K238A aerolysin nanopore. (a) Single channel recording of K238A aerolysin nanopore without addition of any polymers. (b) Raw current trace of polymer ‘00000’ measurement. No signals were observed during the single-channel recording. The concentration of ‘00000’ in the chamber is 100 μmol. (c) Raw current trace of ‘11111’ measurement. There are some signals, but the blockade amplitude and dwell time of signals are too short to decode the bits. The concentration of ‘11111’ in chamber is 100 μmol.

FIG. 7: The K-mean clustering of events of AA00100AA polymer, the clusters of showing a five-level signal were highlighted by the background.

FIG. 8: Signal processing and deep learning workflow.

FIG. 9: Selection percentage versus accuracy obtained from deep learning approach of polymers AA10000AA, AA01000AA, AA00100AA, AA00010AA and AA00001AA.

FIG. 10: Confusion matrix of polymers containing bit-1 at different position: columns represent true polymer from the test set while rows are the polymers that machine learning assigned them to. In an ideal case, it would be a diagonal matrix.

FIG. 11: Confusion matrix of 30 tested polymers. The averaged accuracy is 77.8%.

FIG. 12: Decoding polymer information by wild type aerolysin pore, K238N, E254A and K238Q aerolysin pore mutants.

Wt: AA00200AA polymer (‘2’ is the non-zero bit depicted in FIG. 5, labelled as ‘2’), voltage: 100 mV; K238N mutant: AA00200AA polymer, voltage: 100 mV; E254A mutant: AA00200AA polymer, voltage: 140 mV; K238Q mutant: AA00100AA polymer (‘1’ is the non-zero bit depicted in FIG. 5, labelled as ‘1’), voltage: 100 mV.

FIG. 13: A) Raw current trace after addition of AA00100AA polymer in single MspA system. (B) A typical event and the relative percentage in total events.

FIG. 14: Illustration of single-channel recording setup and reading of polymers encoding single-bit information using an aerolysin pore. Magnification of one single event for AA00100AA (1), AA00200AA (2) and AA00300AA (3) polymers as described in FIG. 5.

### DETAILED DESCRIPTION OF THE INVENTION

The subject-matter herein described will be clarified in the following by means of the following description of those aspects which are depicted in the drawings. It is however to be understood that the subject matter described in this specification is not limited to the aspects described in the following and depicted in the drawings; to the contrary, the scope of the subject-matter herein described is defined by the claims. Moreover, it is to be understood that the specific conditions or parameters described and/or shown in the following are not limiting of the subject-matter herein described, and that the terminology used herein is for the purpose of describing particular aspects by way of example only and is not intended to be limiting.

Unless otherwise defined, technical and scientific terms used herein have the same meaning as commonly understood by one of ordinary skill in the art to which this invention belongs. Further, unless otherwise required by the context, singular terms shall include pluralities and plural terms shall include the singular. The methods and techniques of the present disclosure are generally performed according to conventional methods well known in the art and as described in various general and more specific references that are cited and discussed throughout the present specification unless otherwise indicated. Further, for the sake of clarity, the use of the term “about” is herein intended to encompass a variation of +/— 10% of a given value.

The following description will be better understood by means of the following definitions.

As used in the following and in the appended claims, the singular forms “a”, “an” and “the” include plural referents unless the context clearly dictates otherwise. Also, the use of “or” means “and/or” unless stated otherwise. Similarly, “comprise”, “comprises”, “comprising”, “include”, “includes” and “including” are interchangeable and not intended to be limiting. It is to be further understood that where for the description of various embodiments use is made of the term “comprising”, those skilled in the art will understand that in some specific instances, an embodiment can be alternatively described using language “consisting essentially of” or “consisting of.”

In the frame of the present disclosure, the expression “operatively connected” and similar reflects a functional relationship between the several components of the device or a system among them, that is, the term means that the components are correlated in a way to perform a designated function. The “designated function” can change depending on the different components involved in the connection. A person skilled in the art would easily understand and figure out what are the designated functions of each and every component of the device or the system of the invention, as well as their correlations, on the basis of the present disclosure.

The term “nucleotide” refers to a molecule that contains a nitrogen—containing heterocyclic base (also referred to as “nucleobase”), a sugar or a modified sugar and one or more phosphate groups. For example, in some embodiments, a nucleotide can be a deoxynucleotide triphosphate (dNTP). The term “non-natural nucleotide” as used herein refers to a nucleotide that obeys Watson—Crick base pairing but has a modification that can be detected. By way of example, but not limitation, such a modification can be a functional group attached to the nucleobase such as a methyl group on methylcytosine.

As used herein, the terms “nucleic acid molecule,” “nucleic acid sequence,” “nucleic acid fragment,” “oligonucleotide” and “polynucleotide” are used interchangeably and refer to biopolymers that are made from nucleotides as monomer units. The nucleotide monomers link up to form a linear sequence of the nucleic acid polymer. Nucleic acids encompassed by the present disclosure can include deoxyribonucleic acid (DNA), ribonucleic acid (RNA), cDNA or a synthetic nucleic acid known in the art, such as glycerol nucleic acid (GNA), threose nucleic acid (TNA), locked nucleic acid (LNA) or other synthetic polymers with nucleotide side chains, or any combination thereof. For the sake of easiness, peptide nucleic acids (PNAs), artificially synthesized polymer similar to DNA or RNA, are also included into the definition of oligonucleotides according to the invention.

Nucleotide subunits of nucleic acids can be naturally occurring, artificial, or modified. As indicated above, nucleotide typically contains a nucleobase, a sugar, and at least one phosphate group. The nucleobase is typically heterocyclic. Suitable nucleobases include the canonical purines and pyrimidines, and more specifically adenine (A), guanine (G), thymine (T) (or typically in RNA, uracil (U) instead of thymine (T)), and cytosine (C). The sugar is typically a pentose sugar. Suitable sugars include, but are not limited to, ribose and deoxyribose. The nucleotide is typically a ribonucleotide or deoxyribonucleotide. The nucleotide typically contains a monophosphate, diphosphate or triphosphate. These are generally referred to herein as nucleotides or nucleotide residues to indicate the subunit. Without specific identification, the term nucleotides, nucleotide residues, and the like, is not intended to imply any specific structure or identity.

As indicated above, the nucleic acids of the present disclosure can also include synthetic variants of DNA or RNA. “Synthetic variants” encompasses nucleic acids incorporating known analogs of natural nucleotides/nucleobases that e.g. can hybridize to nucleic acids in a manner similar to naturally occurring nucleotides. Exemplary synthetic variants include peptide nucleic acids (PNAs), phosphorothioate DNA, locked nucleic acids, and the like. Modified or synthetic nucleobases and analogs can include, but are not limited to, 5-Br-UTP, 5-Br-dUTP, 5-F-UTP, 5-F-dUTP, 5-propynyl dCTP, 5-propynyl-dUTP, diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N 6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylam inomethyluracil, 5-methoxyaminomethyl-2-thiouracil, beta-Dmannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Persons of ordinary skill in the art can readily determine what base pairings for each modified nucleobase are deemed a base-pair match versus a base-pair mismatch.

The term “payload” refers to the actual body of data for transmission or for storage or computation. For example, in nucleic acid memory storage, the payload is encoded in the specified nucleotide sequence. The terms “desired data”, “desired information” or “desired media” are used interchangeably to specify the payload information that is contained in the bit stream encoded sequence within a given memory object.

The term “bit” is a contraction of “binary digit”. Commonly “bit” refers to a basic capacity of information in computing and telecommunications. A “bit” conventionally represents either 1 or 0 (one or zero) only, though other higher-order codes can be used with e.g. 2, 4, 6, 8 or more different unit possibilities at every position.

The term “bit stream encoded sequence” as used herein relates to any natural or synthetic sequence-controlled polymer sequence that encodes for data to be stored in a so-called “bitstream-format media”. A “bitstream format” is the format of the data found in a stream (or sequence) of bits used in a digital communication or data storage application. For example, when nucleic acid is used to store data, the “bit stream encoded sequence” is the nucleic acid sequence, either synthetically obtained or naturally occurring, that corresponds to the data that is encoded in a bitstream format.

The terms “sequence-controlled polymer”, “sequence-defined polymer”, “sequence-specific polymer” or “sequence-controlled macromolecule”, as used herein, refer to a macromolecule that is composed of two or more distinct monomers sequentially arranged in a specific, non-random manner, as a polymer “chain”. The arrangement of the two or more distinct monomers constitutes a precise molecular “signature”, or “code” within the polymer chain, particularly in the payload of the molecules of the present disclosure. Sequence controlled polymers can be biological polymers (i.e., biopolymers), or synthetic polymers. Exemplary sequence-controlled biopolymers include natural and/or synthetic nucleic acids, polypeptides or proteins, linear or branched carbohydrate chains, or other sequence controlled polymers that encode a format of information. Exemplary sequence controlled polymers are described in Lutz et al., Science, 341, 1238149 (2013).

As used herein, a “header” refers to supplemental data placed at the beginning of a block of data being stored or transmitted. In the frame of the present disclosure, a header refers to a molecular header, i.e. a supplemental molecular signature, such as a monomer or a polymer, which is placed at the beginning of a sequence-controlled polymer payload storing information to be transmitted. In the same way, a “footer” refers herein to supplemental data placed at the end of a block of data being stored or transmitted. In the frame of the present disclosure, a footer refers to a molecular footer, i.e. a supplemental molecular signature, such as a monomer or a polymer, which is placed at the end of a sequence-controlled polymer payload storing information to be transmitted. Molecular headers and footers according to the invention can preferably but not exclusively be nucleic acid units selected from a list comprising a mononucleotide, a dinucleotide and a nucleic acid sequence such as an oligonucleotide or a polynucleotide. Additionally or alternatively, molecular headers and footers can be other kind of monomers or polymers composed therefrom such as amino acids, oligo- or polypeptides, linear or branched carbohydrate or carbohydrate chains, as well as synthetic chemical entities, or synthetic variants of any of the foregoing.

The term “molecular data storage medium” refers to an object that includes a bit stream-encoded sequence-controlled polymer as a payload of information, at least one header and at least one footer as defined. The bit stream-encoded sequence includes a discrete piece of data, and the at least one header and at least one footer enable selection, organization, and/or isolation of the molecular data storage medium. In some embodiments, molecular data storage media include bitstream-encoded sequence in the form of a continuous stretch of sequence-controlled polymer. In some embodiments, molecular data storage media include discontinuous segments of sequence.

A “nanopore” is any structure comprising and/or defining a pore having a diameter of less than 1 micron, typically between 1 and 20 nm in diameter, for example between 2 and 5 nm in diameter. As a way of example for the sake of providing reference dimensions, single stranded DNA can pass through a 2 nm nanopore, whereas double stranded DNA can pass through a 4 nm nanopore. Having a very small nanopore, e.g., 2-5 nm, allows a biomolecule such as DNA to pass through, but not larger molecular entities such as proteinaceous complexes or enzymes, thereby allowing for controlled passage of polymeric biomolecules or charged polymers in general.

Different types of nanopores are known. For example, biological nanopores are formed by assembly of (a) pore-forming protein(s) in a membrane such as a lipid bilayer. For example, α-hemolysin and similar protein pores are found naturally in cell membranes, where they act as channels for ions or molecules to be transported in and out of cells, and such proteins can be repurposed as nanochannels. Solid-state nanopores are formed in synthetic materials such as silicon nitride or graphene, by configuring holes in the synthetic membrane, e.g. using feedback controlled low energy ion beam sculpting (IBS) or high energy electron beam illumination. Hybrid nanopores can be made by embedding a pore-forming protein in synthetic materials.

Where there is a mean for applying an electrical potential at either end or either side of a nanopore via e.g. electrodes, a current flow across the nanopore may be established through the nanopore via an electrolyte media. Electrodes may be made of any conductive material, for example silver, gold, platinum, copper, titanium dioxide, for example silver coated with silver chloride. The flow of materials across a nanopore may also be regulated by electrodes; for example, as biomolecules are electrically charged, or may be electrically charged depending on some factors such as the pH of the medium they are in (e.g., DNA and RNA are negatively charged in many buffer media), they will be drawn to a positively charged electrode upon application of an electrical voltage across the nanopore. In the event a polymer, such as a sequence-controlled polymer, passes through the nanopore, the change in electric potential, capacitance or current across the nanopore caused by the partial blockage of said nanopore can be detected and used to identify the sequence of monomers in the polymer, wherein different monomers can be distinguished by their different sizes and/or electrostatic potentials.

Methods for configuring a solid state nanopore, a biological nanopore or a hybrid nanopore in membranes or substrates are known in the art, a review of which can be found for instance in Haque, Farzin et al. “Solid-State and Biological Nanopore for Real-Time Sensing of Single Chemical and Sequencing of DNA” Nano today vol. 8, 1 (2013): 56-74, incorporated herein in its entirety by reference.

The terms “membrane”, “film” or “thin film” can be used interchangeably and relate to the thin form factor of an element of the device of the invention. Generally speaking, a “membrane”, “film” or “thin film” as used herein relate to a layer of a material having a thickness much smaller than the other dimensions, e.g. at least one fifth compared to the other dimensions. Typically, a film is a solid layer having a first surface and an opposed second surface, with any suitable shape, and a thickness generally in the order of nanometers or micrometers, depending on the needs and circumstances, e.g. the manufacturing steps used to produce it. In some embodiments, films according to the invention have a thickness comprised between 0.1 nm to 500 μm, such as between 0.3 and 10 nm, between 1 and 50 nm, between 20 and 100 nm, between 200 and 500 nm, between 50 nm and 1 μm, between 1 and 50 μm, between 50 μm and 150 μm, 100 μm and 500 μm or between 200 μm and 500 μm.

In embodiments of the invention, a membrane or thin film can be made of a silicon material, for example silicon dioxide or silicon nitride. Silicon nitride (e.g., Si3N4) is especially desirable for this purpose because it is chemically relatively inert and provides an effective barrier against diffusion of water and ions even when only a few nm thick. Silicon dioxide is also useful, because it is a good surface to chemically modify. Alternatively, in certain embodiments, a membrane or thin film may be made in whole or in part out of materials which can form sheets as thin as a single molecule (sometimes referred to as “single layered” membrane, “monolayer” membrane or “2D” and “two dimensional” sheet or membrane), for example and without limitstion: graphene; GaS; GaSe; GaTe; MX₂ type of dichalcogenides where M=Mo, Nb, Ni, Sn, Ti, Ta, Pt, V, W, or Hf and X=S, Se, or Te; M₂X₃ type of trichalcogenides where M=As, Bi, or Sb and X=S, Se, or Te; MPX₃ where X=S or Se; MAX₃ where A=Si or Ge and X=S, Se, or Te; and alloy sheets like MₓM′₁₋ₓS₂, as well as combinations of any of the foregoing. Accordingly, suitable materials include molybdenum disulfide (MoS₂), molybdenum diselenide (MoSe₂), molybdenum ditelluride (MoTe₂), tungsten disulfide (WS₂), tungsten diselenide (WSe₂), tungsten ditelluride (WTe₂), chromium disulfide (CrS₂), chromium diselenide (CrSe₂), chromium ditelluride (CrTe₂), gallium arsenide, germanium, boron nitride (hBN) and gallium indium phosphide.

A “two-dimensional” or “2D” layer, sheet, polymer, film, membrane and the like is a sheet-like, macromolecule of elements or crystal having a thickness in the order of a single molecule (monomolecular) layer, i.e. of a few nanometers or less, and are therefore not retrievable in nature as free-standing structures. The most known example of a two-dimensional crystal is graphene, an individual, atomically thin layer or sheet of graphite. However, in a broader sense, a 2D structure may comprise more than one monolayer, such as two or three stacked monomolecular layers, and still be considered as two-dimensional in nature. Two-dimensional materials, sometimes also referred to as layered materials, may comprise laterally connected repeat units (monomers) or may be composed of a single or few atomic elements. These materials have found use in applications such as photovoltaics, semiconductors, electrodes and water purification, to cite a few. Layered combinations of different 2D materials are generally called van der Waals heterostructures, and are contemplated in the frame of the present invention.

The term “unit” as used herein refers to a basic element identical or equivalent in function or form with other elements of the same kind, and by comparison with which any other quantity of the same kind is measured or estimated. For instance, when referring to one unit of a chemical species, it is herein meant the single element of said chemical species that forms a base unity of measure to determine the nature of said chemical species. For instance, a nucleic acid unit can be a nucleotide, a dinucleotide, an oligonucleotide such as a sequence of 3, 4, 5, 6 or more nucleotides, a polynucleotide etc., whereas a peptide unit can be one amino acid, a dipeptide, an oligopeptide, a polypeptide etc. The same is true for any kind of chemical species mutatis mutandis, as well as variants thereof. Units according to the invention can be also represents bits in a bit stream-encoded sequence-controlled polymer payload.

According to a main aspect, the present invention discloses a molecular data storage medium comprising:
a header and a footer, each comprising or consisting of at least one unit of a first chemical species; and
a sequence-controlled polymeric chain of a second chemical species located between said header and said footer, said polymeric chain encoding for a desired bitstream-format media.

This first aspect of the invention is based on the consideration and intuition that a molecule designed as a data storage medium typically used in information technology is much more convenient when translated into a molecular data storage setting. In particular, the present inventors designed and synthesized a “hybrid” molecule comprising a payload carrying an information to be stored and decoded operatively linked to an upstream header and a downstream footer, wherein the payload comprises a polymeric chain of a chemical species different from the chemical species forming both the header and the footer. This design and implementation allows some technical and functional advantages when it comes to a molecular data storage and decoding approach. Contrary to the approaches exploited in the prior art, where typically nucleic acid molecules have been used and declined in several possible ways (including 3D structures and non-classical folding, coupling with luminescent labels, modification with functional or bulky groups etc.), or in which nucleic acids and amino acids have been used in the same molecule to have some technical advantage, the presence of a header and a footer which are chemically distinguished from the sequence-controlled polymeric payload allows to 1) direct and orientate the molecular data storage medium towards a decoding spot including a nanopore 2) easily and advantageously synthesize the molecule with readily available and low-cost synthesis approaches and 3) easily distinguish, thanks to their different chemical nature, the encoded data of the payload vis-à-vis the header and the footer, thereby facilitating the decoding of the information whenever needed.

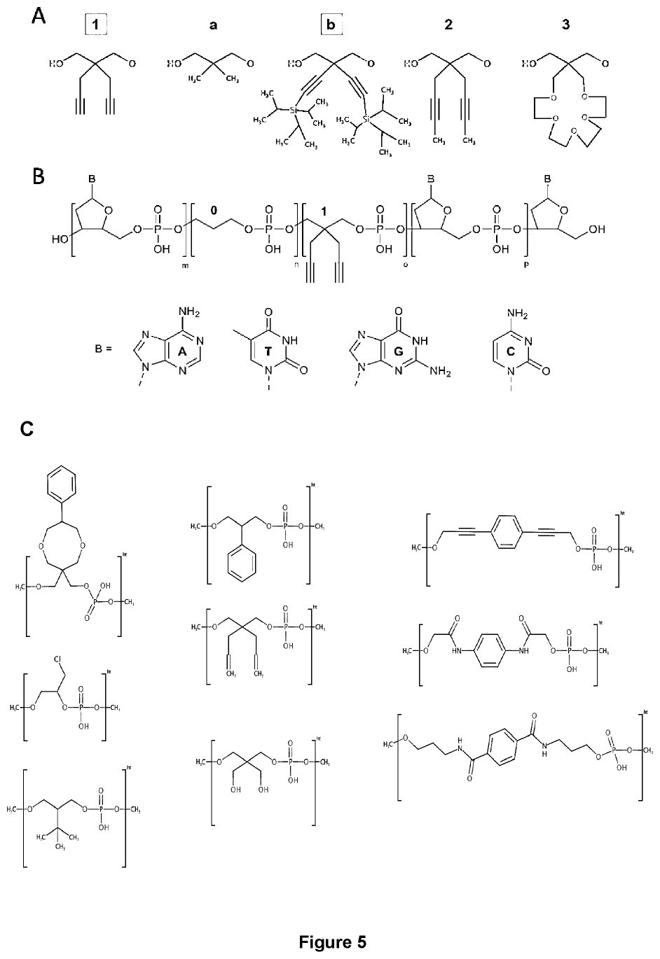

In some embodiments, the header and the footer each comprise or consist of at least one nucleic acid unit as defined before, and the sequence-controlled polymeric chain payload comprises or consists of a non-nucleic acid polymer chain. Non-nucleic acid polymer chain may include amino acids, oligo- or polypeptides, synthetic monomers or polymers, linear or branched carbohydrate chains and the like. In still another embodiment, the sequence-controlled polymeric chain payload comprises or consists of a non-natural nucleic acid polymer chain. The inventors have implemented a series of such sequence-controlled polymeric chains, tailoring in particular the constituting monomers and their chemistry in order to have optimized performances when decoding the payload through a nanopore-based device. Some exemplary monomers are depicted in FIGS. 5a and c, whereas a general molecular structure of one embodiment of the molecular data storage medium according to the invention is shown in FIG. 5b. The depicted nucleic acid units feature chemically-modified nucleotide monomers suitable in the frame of the present invention. Methods for synthesizing sequence-controlled polymer, as well as possibly headers and footers according to the invention, are readily available to a person skilled in the art, a review of which being provided for instance in Lutz et al., Science, 341, 1238149 (2013), incorporated herein in its entirety by reference. In one embodiment, sequence-controlled polymeric chain payloads are synthesized using a phosphoramidite chemistry approach as described for instance in Al Ouahabi et al., Journal of the American Chemical Society 2015, 137 (16), 5629-5635, incorporated herein in its entirety by reference.

The header and the footer have in embodiments of the invention the same chemical nature, i.e. they are composed of the same chemical species. In embodiments, the header and the footer have the same length. In embodiments, the header and the footer are composed of the same number of units. In embodiments, the units of the header and the footer are the same. As a way of example, the header and the footer may comprise one or more mononucleotide, dinucleotide, oligonucleotide or polynucleotide units, such as for instance a dinucleotide unit (e.g. AA, CC, GG, TT etc.). In embodiment envisaging a header and/or a footer comprising nucleic acid units, said nucleic acid may contain only two base types and does not contain any bases capable of self-hybridizing, e.g., wherein the DNA comprises adenines and guanines, adenines and cytosines, thymidines and guanines, or thymidines and cytosines.

In embodiments of the invention, said header and/or said footer may comprise a unit of a first chemical species having a sequence complementary to the unit of a first chemical species of a header and/or a footer of a second molecular data storage medium. The complementarity of sequences in headers and/or footers may allow the association of molecular data storage media of the invention into larger super-structures based on a pool of memory media, enable physical association in supra-memory blocks for networking and/or spatially segregating blocks of related information, in a way as to for instance allow to a decoding system rapid retrieval of said pool of memory information. Typically, assembly occurs through complementary sequences on overhangs, through a bridging oligonucleotide (splint strand) in case said first chemical species is a nucleic acid, or through protein or chemical adducts to overhangs. The super-structured molecular data storage media can be specifically dissociated and re-grouped by using external signals as desired by the user. Exemplary external signals used to control dissociation include changing the pH, lowering the salt concentration in a molecule-containing buffer, increasing the temperature, applying an electro-magnetic radiation, toe-hold strand displacement, complementary strand excess, or enzymatic release by restriction nucleases, nickases, helicases, resolvases, releasing using UV-sensitive linker, using CRISPR/Cas9 and guide RNAs, or any combination thereof.

In embodiments, the molecular data storage media according to the invention comprise sequence-controlled polymeric chain payloads in which each monomer composing the same encodes for one or more bits of a bitstream-format media, such as 2 bits/monomer, 3 bits/monomer or higher. In one embodiment, data storage media according to the invention comprise sequence-controlled polymeric chain payloads in which each monomer composing the same encodes for a single bit of a bitstream-format media. Advantageously, in embodiments said sequence-controlled polymeric chain is composed of a sequence of two or more types of monomers, i.e. two distinct monomers of the same chemical species, thereby having a plurality of monomers arranged in sequence to correspond to a binary code. The use of only two, distinct monomers, one representing bit-0 and the other representing bit-1, facilitates at the same time the synthesis of the polymers, the encoding and the decoding of information, inter alia, thereby permitting operations similar to bitstream format memory data typically used in information technology. The use of more than two monomers is also possible, as it may improve the storage density of the molecular data storage media. The bit stream may also be improved by the use of error-correcting codes and data compression methods.

As it will be apparent, a second aspect of the present invention concerns a method for encoding a bitstream-format information in a molecular data storage medium, comprising the steps of:
providing a desired digital media in a bitstream-format; and
converting said bitstream-format media into a molecular data storage medium according to the invention. The conversion step typically comprises synthesizing a payload of a desired data, this being the sequence-controlled polymeric chain of a molecular data storage medium, so that every monomer or group of monomers of said polymeric chain encodes for a bit or group of bits of said bitstream-format media.

The present invention is further directed to systems and methods for digital data decoding, said digital data being encoded in a molecular data storage medium according to the invention. In particular, the invention features a system adapted and configured for reading data stored in a molecular data storage medium. Even more particularly, the invention features a nanopore-based device adapted and configured for reading data stored in a molecular data storage medium, said nanopore-based device comprising:
a reservoir;
a membrane located on or within said reservoir in a way to split said reservoir in two facing chambers;
means for providing a voltage operatively connected to said reservoir; and
at least one nanopore spanning across the thickness of said membrane.

Preferably, the device further comprises means for recording and analysing an electrical current. The membrane can be either a solid state membrane or a biological membrane, such as a lipid bilayer. In embodiments of the invention, the membrane comprises an array of nanopores, and the device can be accordingly configured to record and analyse an electrical current obtainable from more than one nanopore.

The design and technical features of the device is tightly linked to, and based upon, a method for decoding a bitstream-format information encoded in the molecular data storage medium according to the invention, which represents a further aspect of the present disclosure. In one embodiment, said method comprises the steps of:
providing a nanopore-based device for reading data stored in a molecular data storage medium according to the present disclosure;
providing a molecular data storage medium according to the invention in a suitable buffer and in one chamber of the nanopore-based device reservoir;
providing a transmembrane voltage to said reservoir, thereby allowing the passage of said molecular data storage medium through said nanopore from one chamber to the opposed chamber of the reservoir; and
recording and analysing an electrical current during the passage of said molecular data storage medium through said nanopore, thereby decoding a bitstream-format information.

The device of the invention comprises at least two chambers separated by one or more nanopores, wherein each chamber is configured to comprise an electrolytic fluid and one or more electrodes to draw an electrically charged polymer according to the invention from one chamber to another. The device may optionally be configured with functional elements to guide, channel and/or control the molecular data storage medium of the invention, it may optionally be coated or made with materials selected to allow smooth molecule flow, and it may comprise for instance circuit elements to provide and control electrodes proximate to the nanopores. For example, the one or more nanopores may optionally each be associated with electrodes which can control the movement of the polymer though the nanopore and/or detect changes in electrical potential, current, resistance or capacitance at the interface of the nanopore and the polymer, thereby detecting the sequence of the polymer as it passes through the one or more nanopores. As the polymer passes through the nanopore, the change in electrical potential, capacitance or current across the nanopore caused by the partial blockage of the nanopore can be detected and used to identify the sequence of monomers in the polymer, as the different monomers can be distinguished by their different sizes and electrostatic potentials.

Accordingly, the methods of the invention involve the measuring of a current passing through the pore as the substrate, such as a target molecular data storage medium, moves with respect to the pore. Suitable conditions for measuring ionic currents through transmembrane protein pores are known in the art. The method is typically carried out with a voltage applied across the membrane and pore. It is possible to increase discrimination between different monomers of the substrate by a pore by e.g. using an increased applied potential.

The current needed to move a charged polymer through the nanopore depends on, e.g., the nature of the polymer, the size of the nanopore, the material of the membrane containing the nanopore and/or the salt concentrations, and so need to be optimized to the particular system depending on the needs and circumstances. In the case of the hybrid polymeric molecules as used in the present invention, examples of voltage and current would be, e.g., between −300 and +300 mV, typically between 80 and 140 mV, and between −250 and 250 pA, e.g., between 40 and 120 pA, with salt concentrations on the order of 0.1 and 10 M.

The methods are typically carried out in the presence of any charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), lithium chloride (LiCl), sodium chloride (NaCl) or caesium chloride (CsCl) is typically used. The salt concentration may be at saturation. High salt concentrations provide a high signal to noise ratio and allow for currents indicative of the presence of a nucleotide to be identified against the background of normal current fluctuations.

The methods are typically carried out in the presence of a buffer. In the exemplary apparatus discussed above, the buffer is present in the aqueous solution in the chamber. Any suitable buffer may be used in the method of the invention. Typically, the buffer is HEPES. Another suitable buffer is Tris-HCl buffer. The methods are typically carried out at a pH of from 3.0 to 12.0, preferably about 7.5.

The methods may be carried out at temperatures from 0° C. to 100° C., such as from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

In one embodiment, the step of recording and analysing an electrical current comprises measuring a relative current distribution I/I₀, wherein I₀ is the value of the open nanopore current and I is the residual current value during the passage of said molecular data storage medium through said nanopore.

In order to implement the methods of the invention (particularly a method for decoding a bitstream-format information encoded in a molecular data storage medium), the system may comprise an operatively coupled computing device configured to control the operation of the system, said computing device comprising a memory and a processing unit encoding instructions that, when executed, cause the processing unit to control at least one of the means to provide a voltage and the means for recording and analyzing an electrical current. The computing device may include one or more processing units and computer readable media. Computer readable media includes physical memory such as volatile memory (such as RAM), non-volatile memory (such as ROM, flash memory, etc.) or some combination thereof. Additionally, the computing device can include mass storage (removable and/or non-removable) such as a magnetic or optical disks or tape. An operating system and one or more application programs can be stored on the mass storage device. The computing device can further include input devices (such as a keyboard and mouse) and output devices (such as a monitor), if needed.

In sequencing devices known in the art, typically a nanopore is used in a fluid-filled cell to read usually DNA data by measuring a change in current as the DNA passes through the nanopore, which are typically in the range of nano-amps. Accordingly, it is very difficult to reliably and repeatably detect such small changes, as they are difficult to distinguish over typical background noise. The difficulties are further enhanced in that charged polymers like DNA can move through a nanopore at the rate of approximately one million bases per second, which is too fast to be read accurately using means known in the art, requiring the use of protein nanopores which slow the passage of DNA through the nanopore itself, and which were considered impractical for reading data.

To the contrary, the present inventors proved able to manage the very rapid movement of the molecular data storage polymer of the invention while getting an accurate reading thereof distinct from the noise in the system. In particular, the inclusion in one embodiment of the device of the invention of at least one biological, macromolecular nanopore selected from a list comprising pore-forming toxins and mutated pore-form toxins, resulted in an astoundingly precise and reliable measurement of tiny current changes during the passage of the hybrid molecule of the invention through said nanopores. Non-limiting examples of suitable biological, macromolecular nanopore comprise wild-type or mutated versions of Alpha hemolysin (aHL), Mycobacterium smegmatis porin A (MspA) and aerolysin, to cite some. In a preferred embodiment of the invention, said pore-forming toxin and/or mutated pore-form toxin is at least one of an aerolysin pore or a mutated aerolysin pore.

One aspect of the invention relates to the optimization of nanopores used for implementing the methods of the invention for decoding a bitstream-format information encoded into a molecular data storage medium, in parallel with the optimization of the (stereo)chemical nature of the molecular data storage medium. In this sense, as will be better detailed in the Example section herein below, the type and structure of the monomers used in the payload of the molecular data storage medium has “evolved” together with the sensing interface of the nanopores.

The inventors have developed in the past a series of aerolysin mutants that have been rationally designed and studied, using molecular modelling and simulation based on recent aerolysin structures and models, in order to alter the interaction between an aerolysin monomer and an analyte such as a polynucleotide, polypeptide or small molecules such as ions. Pores comprising said mutant monomers have an enhanced ability to interact with a substrate analyte such as polynucleotides, polypeptide and small molecules, and therefore display improved properties for estimating the characteristics of, such as the sequence of, said analytes. The aim of this aerolysin mutation process was to increase a current blockage difference/variance (with respect to the basal current of the open pore) in order to better discriminate different monomers of a polymer in a nanopore sensing approach. Such aerolysin mutants, including pores and various constructs obtainable therefrom, are described in European patent application 19 197 435.1, incorporated herein in its entirety by reference.

However, the inclusion of mutated aerolysin pores into systems configured for decoding digital information was never tested in the past. The use of aerolysin pores in this context has been validated with surprisingly good results when used in combination with the molecular data storage media of the invention.

In particular embodiments of the invention, therefore, the device of the invention exploits the improved sensing abilities of those aerolysin nanopores. A mutant aerolysin pore useful in the frame of the present invention comprises one or more modifications on the aerolysin monomer sequence that change the net positive charge, as well as the size of the pore region formed upon oligomerization of the monomers into a pore-forming structure. Said net charge is increased by e.g. introducing one or more positively charged amino acids and/or by neutralising one or more negative charged amino acids, for instance by substituting one or more negatively charged amino acids with one or more uncharged amino acids, non-polar amino acids and/or aromatic amino acids or by introducing one or more positive charged amino acids adjacent to one or more negatively charged amino acids. The size of the pore is altered by increasing or reducing the steric hindrance of side-chain protruding to the internal lumen of the pore.

A modified aerolysin polypeptide to be used as a monomer in an aerolysin pore generally comprises, consists essentially of or consists of a modified aerolysin amino acid sequence. An amino acid sequence of a wild-type (i.e., native, unmodified) aerolysin monomer polypeptide from Aeromonas hydrophila is provided herein as SEQ ID NO: 1 which corresponds to region or positions 24-493 of the wild type aerolysin protein sequence https://www.ncbi.nlm.nih.qov/protein/P09167.2. Such modifications alter the ability of the aerolysin monomer, assembled in a heptameric pore form, to interact with a polymer such as a polynucleotide, a polypeptide or even another analyte via (i) a steric effect of the aerolysin pore on the interacting substrate, (ii) a net charge alteration of the aerolysin pore and/or (iii) the ability of the aerolysin pore to alter the hydrogen bonds established with an interacting substrate.

Said monomer can comprise or can consist of a polypeptide comprising a modified aerolysin amino acid sequence, wherein said sequence comprises the amino acid sequence of SEQ ID NO: 1 or the amino acid sequence of SEQ ID NO: 2 (representing the mature aerolysin monomer without a C-terminal propeptide, namely positions 24-445 of the wild type aerolysin protein sequence) having one or more amino acid substitutions at one or more positions corresponding to positions 220, 238, 242 and 282. In some additional or alternative embodiments, polypeptides further comprises one or more amino acid substitutions at one or more positions corresponding to positions 216, 222, 244, 246, 252, 254 and 258 of SEQ ID NO: 1 or SEQ ID NO: 2.

Preferably, the amino acid(s) substituted into the mutant aerolysin monomer at positions R220, K238, K242 and R282 are selected from the group comprising asparagine (N), glutamine (Q), arginine (R), glutamic acid (E), leucine (L), lysine (K), cysteine (C), tryptophan (W), histidine (H) or alanine (A).

In embodiments, a mutant aerolysin monomer comprises at least one of the following mutations: R220A/W/K/Q, R282A/E/W, K238A/Q/N/R/W/H, K242A/W as well as any combination thereof. Preferably, the amino acid(s) substituted into the mutant aerolysin monomer at positions D216, D222, D222, K244, K246, E252, E254 and E258 are selected from the group comprising asparagine (N), glutamine (Q), arginine (R), aspartic acid (D) or alanine (A). In embodiments, a mutant aerolysin comprises at least one of the following mutations: D216A/N/Q/R, D222A/N/Q/R, K244A/N/Q/R/D, K246A/N/Q/R/D, E252A/N/Q/R, E254A/N/Q/R, E258A/N/R/Q as well as any combination thereof.

In embodiments of the invention, a mutant aerolysin monomer comprises a substitution on at least one of the following positions of SEQ ID NO: 1 or SEQ ID NO: 2: 220, 238, 242 and 282 (hereinafter referred to “group 1 of mutations”) together with a substitution on at least one of the following positions 216, 222, 244, 246, 252, 254 and 258 (hereinafter referred to “group 2 of mutations”). For example, a mutant aerolysin monomer comprises at least one of the following mutations in group 1 of mutations: R220A/W/K/Q, R282A/E/W, K238A/Q/N/R/W/H, K242A/W, as well as at least one of the following mutations in group 2 of mutations: D216A/N/Q/R, D222A/N/Q/R, K244A/N/Q/R/D, K246A/N/Q/R/D, E252A/N/Q/R, E254A/N/Q/R, E258A/N/R/Q as well as any combination thereof.

A mutant aerolysin pore suitable in the frame of the present invention may comprise at least one polypeptide of SEQ ID NO: 2 (representing the mature aerolysin monomer without a C-terminal propeptide) or a variant thereof having one or more amino acid substitutions at one or more positions corresponding to positions 220, 238, 242, 282, 216, 222, 244, 246, 252, 254 and 258; additionally or alternatively, a homo-oligomeric pore derived from said mutant aerolysin monomer comprising identical mutant monomers and a hetero-oligomeric pore derived from said mutant aerolysin monomer as described herein, wherein at least one of the monomers differs from the others are envisaged in the frame of the invention.

A mutant monomer can be produced using standard methods known in the art. Polynucleotide sequences encoding a mutant monomer may be expressed in a bacterial host cell using standard techniques in the art. The mutant monomer may be produced in a cell by in situ expression of the polypeptide from a recombinant expression vector. The expression vector optionally carries an inducible promoter to control the expression of the polypeptide. The monomer may be made synthetically or by recombinant means. For example, the monomer may be synthesized by in vitro translation and transcription (IVTT). Suitable methods for producing pore monomers are discussed for instance in International Applications WO 2010/004273, WO 2010/004265 or WO 2010/086603.

### EXAMPLES

Aerolysin Nanopores

In the following, non-limiting examples the inventors show that aerolysin pores have the potential to achieve the molecular equivalent of single-base resolution for tailored digital analytes, which in turn allows for single-bit reading accuracy. Using deep learning, the inventors were able to decode digital sequences encoding up to 4-bit information with a high accuracy, while blindly detect the identity and relative concentration of polymer mixtures.

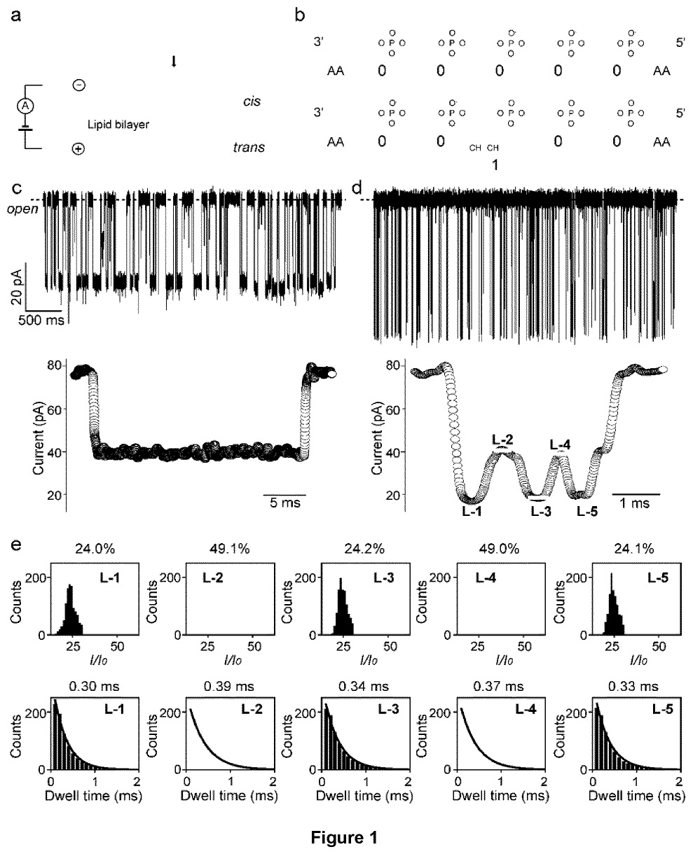

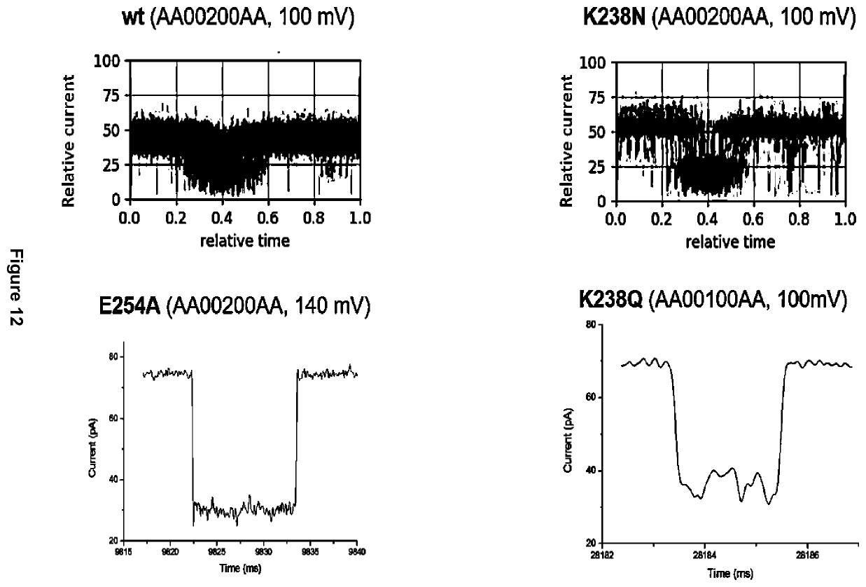

Single-channel recording experiments were performed to analyze polymer translocation using the PFT aerolysin from Aeromonas hydrophila (FIG. 1a). Aerolysin is one of the best characterized among PFTs, it oligomerizes into a heptameric pore that features a novel and unique fold, constituted by two concentric p-barrels held together by hydrophobic interactions. Aerolysin has been proposed to be a promising nanopore sensor, exhibiting high sensitivity for molecular detection, providing excellent current separation and a dwell time range suitable for accurate signal processing. Recently, aerolysin mutants have been rationally designed to further enhance the sensing properties of the wild-type pore; notably, the K238A mutant has shown a significantly enhanced resolution for molecular recognition and turned out to be the most suitable sensing system to detect the tailor-made molecular structure of the digitally-encoded polymers ad hoc developed in this work. However, some additional experiments were conducted with alternative aerolysin mutants and even with wild type pores, as shown in FIG. 12.

These macromolecules are sequence-defined poly(phosphodiester)s prepared by automated phosphoramidite chemistry (FIG. 1b and FIG. 5). The negatively charged backbone of these polymers ensures an efficient capture of polymer in a nanopore and the translocation from the cis (first chamber) to trans (second chamber) compartment of a reservoir under an applied voltage. The polymers were digitally-encoded using two monomers of different molecular structure. In the formed chains, the synthons n-propyl-phosphate and (2,2-dipropargyl)-propyl-phosphate represent bit-0 and bit-1, respectively. It shall be noted that this binary alphabet differs from the one that is usually used for mass spectrometry sequencing. Here, two monomers of markedly-different bulkiness were selected in order to induce different pore current responses. Furthermore, automated phosphoramidite chemistry allows the use of both biological (i.e. natural DNA nucleotides) and non-biological monomers. Thus, in the present work, bio-hybrid macromolecules, composed of the aforementioned non-natural binary alphabet as well as natural nucleotides, were examined for optimal pore translocation. All the digitally-encoded polymers examined in this study are listed in Table 1.

TABLE 1
Characterization of the digital poly(phosphodiester)s.

| Sequence | Short Name | Mass (Da) | m/zₜₕ | m/zₑₓₚ^{a} |
|---|---|---|---|---|
| 00000 | — | 628.0852 | 627.0779^{b} | 627.0761 |
| 11111 | — | 1008.2417 | 1007.2344^{b} | 1007.2339 |
| AA00100CC | — | 1908.3245 | 476.0738^{d} | 476.0744 |
| CC00100AA | — | 1908.3245 | 476.0738^{d} | 476.0738 |
| CC00100CC | — | 1860.3020 | 464.0682^{d} | 464.0681 |
| AA00100TT | — | 1938.3238 | 483.5737^{d} | 483.5735 |
| AA00100GG | — | 1988.3368 | 496.0769^{d} | 496.0773 |
| GG00100AA | — | 1988.3368 | 496.0769^{d} | 496.0774 |
| TT00100AA | — | 1938.3238 | 483.5737^{d} | 483.5737 |
| AA000AA | 0 | 1604.2993 | 533.7591^{c} | 533.7589 |
| AA010AA | 1 | 1680.3306 | 559.1029^{c} | 559.1027 |
| AA0000AA | 00 | 1742.3074 | 579.7619^{c} | 579.7622 |
| AA0010AA | 01 | 1818.3387 | 605.1056^{c} | 605.1054 |
| AA0100AA | 10 | 1818.3387 | 605.1056^{c} | 605.1058 |
| AA0110AA | 11 | 1894.3701 | 630.4494^{c} | 630.4493 |
| AA00000AA | 000 | 1880.3156 | 469.0716^{d} | 469.0712 |
| AA00010AA | 001 | 1956.3469 | 651.1084^{c} | 651.1083 |
| AA00100AA | 010 | 1956.3469 | 651.1084^{c} | 651.1082 |
| AA01000AA | 100 | 1956.3469 | 651.1084^{c} | 651.1080 |
| AA00110AA | 011 | 2032.3782 | 507.0873^{d} | 507.0880 |
| AA01010AA | 101 | 2032.3782 | 507.0873^{d} | 507.0873 |
| AA01100AA | 110 | 2032.3782 | 507.0873^{d} | 507.0877 |
| AA01110AA | 111 | 2108.4095 | 526.0951^{d} | 526.0955 |
| AA000000AA | 0000 | 2018.3238 | 671.7673^{c} | 671.7669 |
| AA000010AA | 0001 | 2094.3551 | 522.5815^{d} | 522.5814 |
| AA000100AA | 0010 | 2094.3551 | 522.5815^{d} | 522.5817 |
| AA001000AA | 0100 | 2094.3551 | 522.5815^{d} | 522.5812 |
| AA010000AA | 1000 | 2094.3551 | 522.5815^{d} | 522.5815 |
| AA000110AA | 0011 | 2170.3864 | 541.5893^{d} | 541.5893 |
| AA001010AA | 0101 | 2170.3864 | 541.5893^{d} | 541.5892 |
| AA001100AA | 0110 | 2170.3864 | 541.5893^{d} | 541.5888 |
| AA010010AA | 1001 | 2170.3864 | 541.5893^{d} | 541.5894 |
| AA010100AA | 1010 | 2170.3864 | 541.5893^{d} | 541.5892 |
| AA011000AA | 1100 | 2170.3864 | 541.5893^{d} | 541.5894 |
| AA001110AA | 0111 | 2246.4177 | 560.5972^{d} | 560.5969 |
| AA010110AA | 1011 | 2246.4177 | 560.5972^{d} | 560.5975 |
| AA011010AA | 1101 | 2246.4177 | 560.5972^{d} | 560.5975 |
| AA011100AA | 1110 | 2246.4177 | 560.5972^{d} | 560.5974 |
| AA011110AA | 1111 | 2322.4490 | 579.6050^{d} | 579.6052 |
| AA0000000AA | 00000 | 2156.3320 | 538.0757^{d} | 538.0759 |
| AA0001000AA | 00100 | 2232.3633 | 557.0836^{d} | 557.0832 |
| AA00000000AA | 000000 | 2294.3402 | 572.5778^{d} | 572.5778 |
| AA00001000AA | 000100 | 2370.3715 | 591.5856^{d} | 591.5854 |

^{a}Values obtained by ESI-HRMS measurements in the negative ion mode;
^{b}Detected as [M − 3H]³⁻;
^{c}Detected as [M − 4H]⁴⁻.

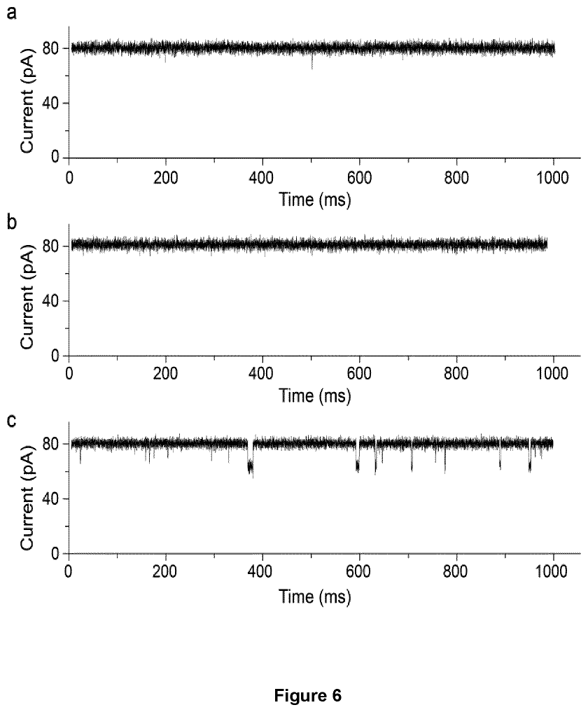

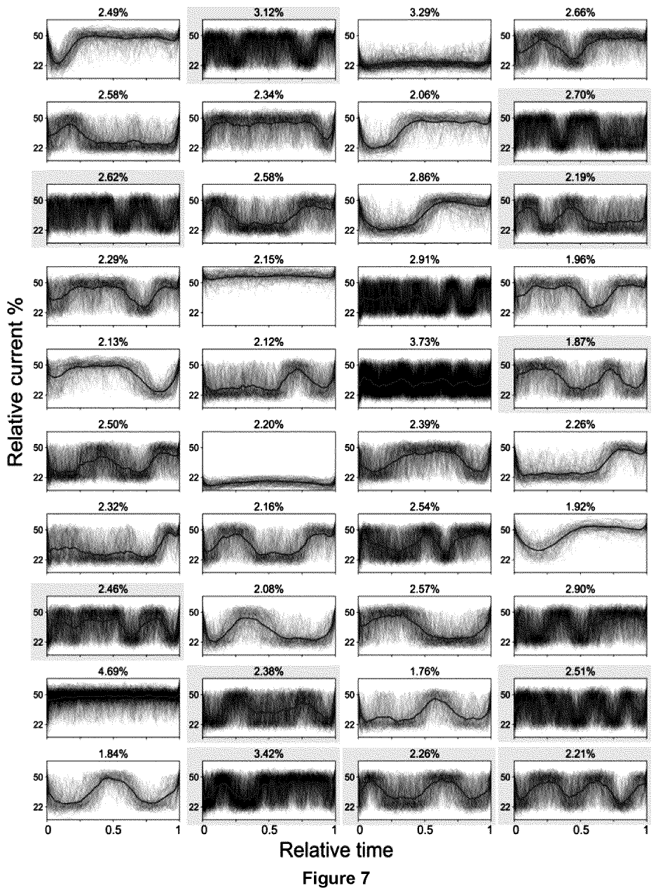

Digital decoding in aerolysin pores was first attempted with copolymers containing only bit-0 and bit-1 monomers. The negatively charged backbone of these polymers ensures efficient translocation, but also speeds up the crossing time that would be too fast to allow decoding. As no signals were observed during the single-channel recording after addition of ‘00000’ polymer in the cis side of the chamber and the signal-to-noise-ratio was too low for ‘11111’ (FIG. 6), the inventors introduced a di-deoxyadenosine at the polymer terminals (‘AA00000AA’, FIG. 1b). This addition created highly detectable current blockade signals (39.0±3.0 pA as mean residual current, with an open pore current of 76.0±3.0 pA, and 18.9±1.3 ms for dwell time), easily identifying the translocation of ‘AA00000AA’ polymers (FIG. 1c). When a bit-1 monomer was inserted to create an ‘A00100AA’ polymer, the additional moiety induced an obvious lowering of the current levels (FIG. 1d). Compared to ‘AA00000AA’, a clear decrease in the mean dwell time (2.8±0.1 ms) was observed and a fraction of the events (˜28%) clearly showed a five-level signal (labeled L-1 to L-5 in FIG. 1d, FIG. 7).

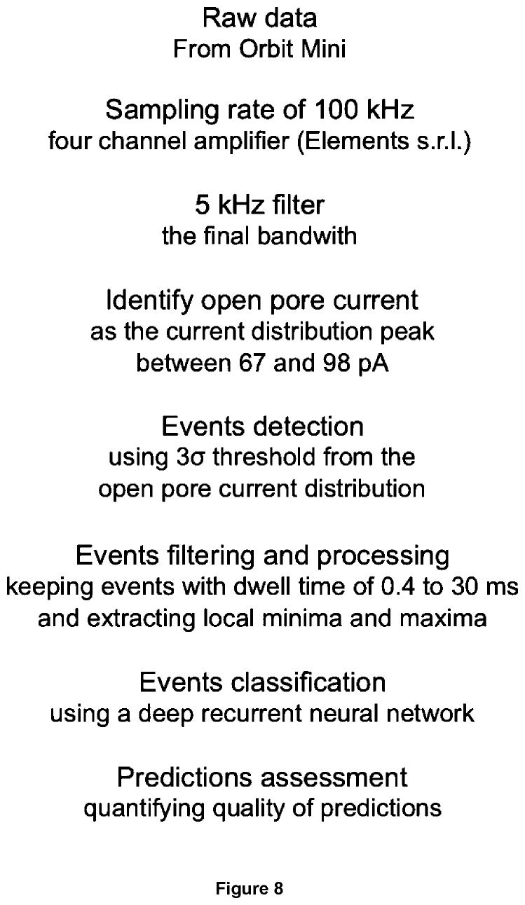

To understand the relationship between the polymer chemical nature and current levels, the inventors collected more than 10′000 blockade current events for statistical analysis (see Methods and FIG. 8). The relative current (I/I₀), fitted as Gaussian distribution, showed 5 distinct levels of values respectively 24.0±3.2%, 49.1±2.4%, 24.2±2.3%, 49.0±2.5% and 24.1±2.4% (FIG. 1e, I₀ is the value of the open pore current and I is the residual current value). The dwell times of each level, fitted with an exponential function, have values of 0.30±0.01, 0.39±0.01, 0.34±0.01, 0.37±0.01, and 0.33±0.01 ms, respectively. The I/10 values of L-2 and L-4 are nearly identical, while L-1, L-3 and L-5 are also quite similar, and all the current states share a similar characteristic dwell time (FIG. 1e). According to previous studies, the inventors found a strong correlation between the physical size of the translocated molecules and I/I₀ values; therefore L-1 and L-5 can be likely interpreted as the blockade caused by the added volume of the di-deoxyadenosine moieties at the two terminals, while similarly L-3 to the bulky (2,2-dipropargyl)-propyl-phosphate (bit-1). The two lighter n-propyl-phosphates flanked between these bulkier groups (FIG. 1b) contribute instead to higher L-2 and L-4 current states. Therefore, the strategy of including nucleotides in informational polymer not only decreased the dwell time but also enhanced the potential resolution of the system to single-bit precision.

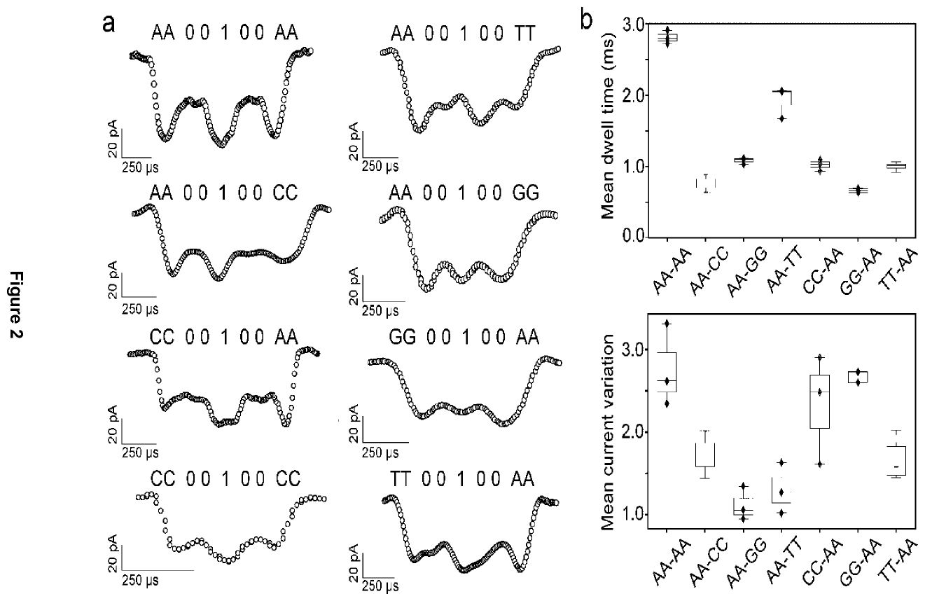

To optimize the polymer design and further understand the influence of the terminal nucleotides for decoding, a series of polymers which replaced the terminal di-deoxyadenosine groups with other types of dinucleotides were tested. According to previous observations, DNA prefers to enter the aerolysin pore from the 3′-terminal (thus all polymers are oriented starting from the 3′-end, FIG. 1b). Translocation events of these polymers showed a qualitatively similar 5-level signal (FIG. 2a). While L-1 values for AA00100AA and AA00100CC are nearly identical, in CC00100AA the relative current is higher, demonstrating eventually that the first current blockade is associated to the 3′ nucleobase and its chemical nature. Among all polymers, L-1 and L-5 of CC00100CC are the highest peaks, which further supports the hypothesis that the first and last current levels are induced by nucleobases at the terminals. Therefore, aerolysin nanopores are able to read not only tailor-made informational polymers, but also different types of DNA bases and their order at the terminals. To systemically evaluate the enhancement of different dinucleotides for separation between bit-1 and bit-0 monomers, the inventors compared the mean dwell time (i.e., longer dwell time allows a more accurate determination of blockade current levels) and mean current variation (i.e., higher variation promotes a higher read accuracy for each bit, see Methods) of all polymers with different terminals (FIG. 2b). As di-deoxyadenosine at both terminals showed the longest dwell time and highest current variation among all polymers, it was chosen as the basic terminal block for the following design.

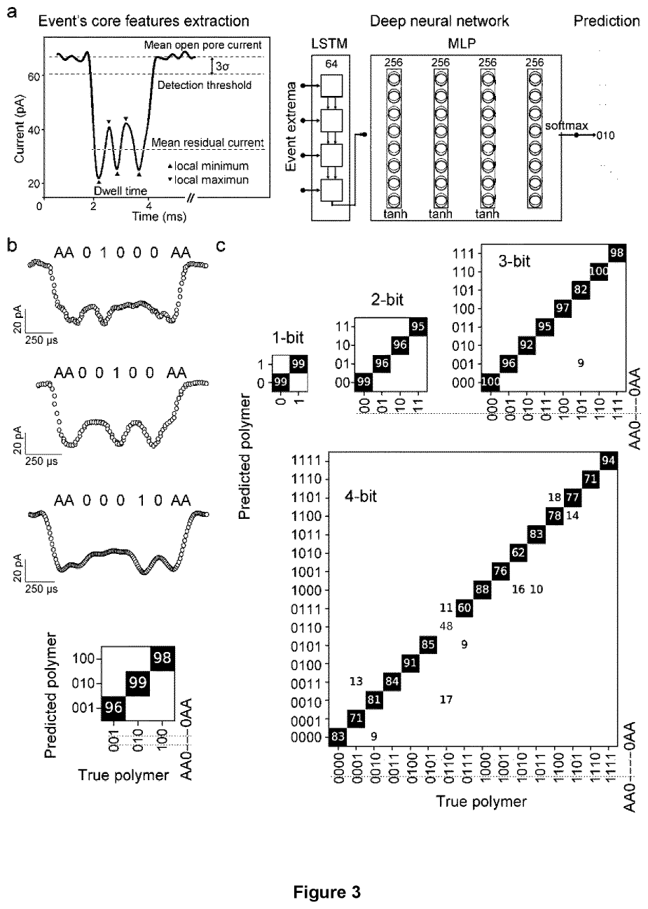

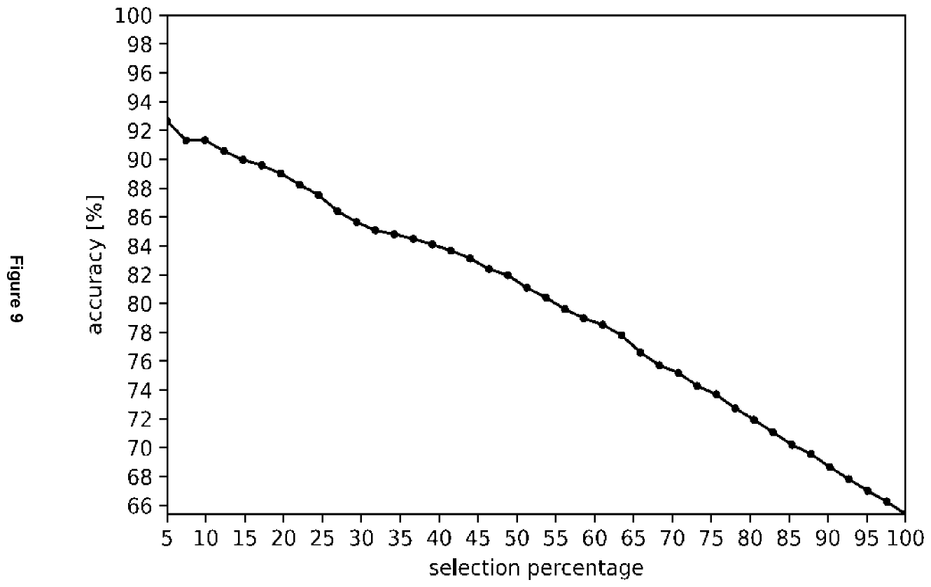

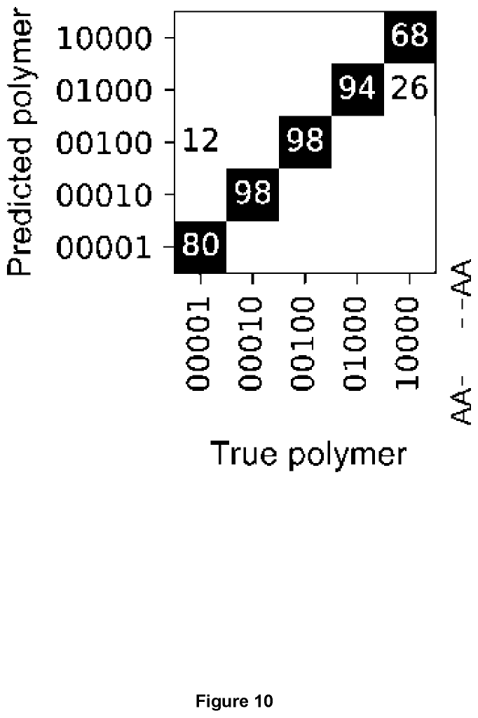

It was then tested the sensitivity of the pore for detecting bit-1 monomers when spanning the 5 available positions along the n-propyl-phosphate backbone (i.e., AA10000AA, AA01000AA, AA00100AA, AA00010AA and AA00001AA). For this task, the inventors developed a deep learning approach to process the current signal, which was able to automatically classify a much larger fraction of events (˜40%) with high accuracy (˜84%, FIG. 9). Long short-term memory (LSTM) recurrent neural network (RNN) was used to read the events local extrema followed by a multilayer perceptron (MLP) to classify the polymers (FIG. 3a). These additional results showed that the detection of bit-1 monomers is difficult when flanking directly the terminal nucleobases (FIG. 10). When only the innermost positions are considered (i.e., as in AA01000AA, AA00100AA, AA00010AA) the resulting accuracy of the neural network is as high as 97.6% (FIG. 3b), therefore these positions were only used for single-bit reading, hereafter indicated in bold (e.g., AA00000AA). Indeed, the inventors tested between AA0- and -0AA flanking terminals polymers encoding for 1 to 4 bits of information generating a library of 30 different polymer sequences (i.e., 2¹+2²+2³+2⁴, FIG. 3c). While each of these 30 polymers were measured by a single nanopore independently, a reading accuracy of 98.7% for 1-bit polymers, 96.4% for 2-bit, 95% for 3-bit and 76.9% for 4-bit was reported.

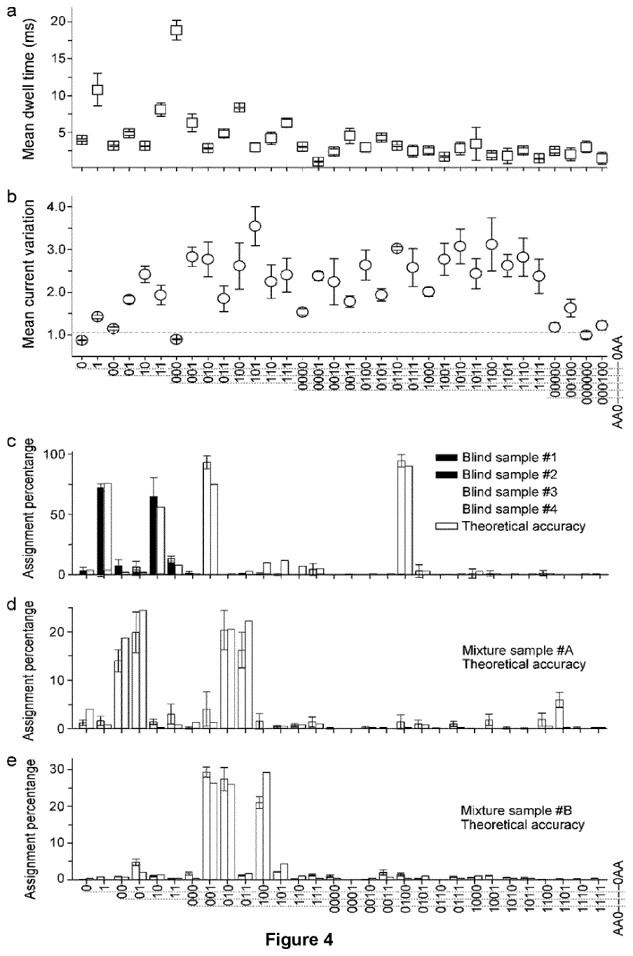

To better evaluate the reading capability of aerolysin nanopores the inventors performed a statistical analysis across N=546 separate nanopore measurements of all 30 polymers (˜6.6M events in total, FIG. 4a). Notably, few 5-bit and 6-bit polymers were also included to expand the polymer landscape. In general, the averaged dwell time decreased as polymer length increased tending nonetheless to a value of 2.5±0.8 ms. In particular, if the total coding length is shorter than 3 bits the dwell time is longer, likely due to the presence of a more abundant negatively charge density per bit, which is steered faster by the applied voltage; while dwell times tend to converge to higher value when the polymer length is longer than 4-bit and charge density is less affected by the terminal groups (FIG. 4a). On the other hand, although 4-bit polymer dwell times decreased, their current variation increased (FIG. 4b), indicating that more information is encoded in the longer polymers.

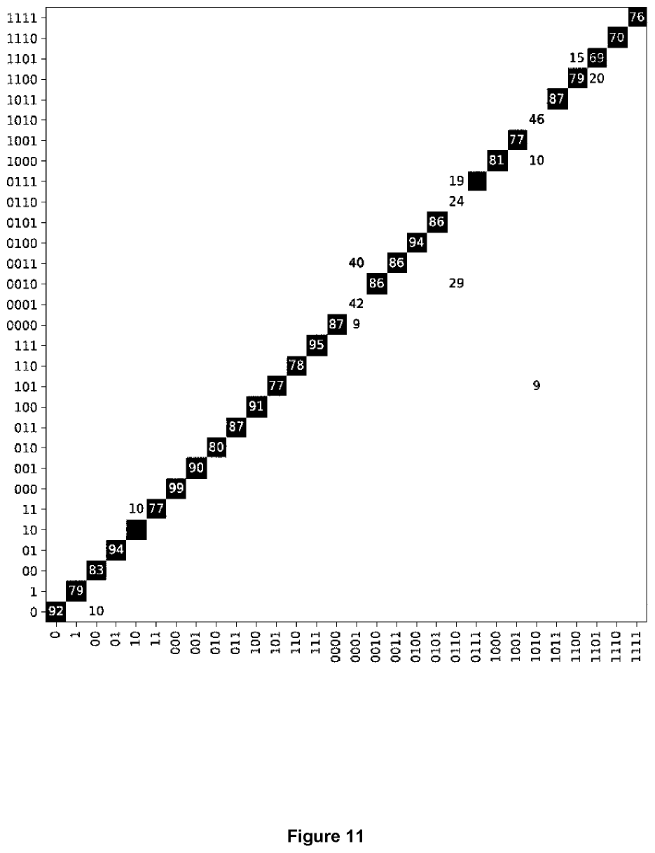

Based on the model generated by deep learning, the expectation is that any item in the library of polymer sequences can be identified directly with high confidence. To test this hypothesis, blind tests was performed to identify the given polymers and their relative concentration when mixed. Following this blind procedure, the inventors were able to correctly detect polymer “AA010AA” among all 30 polymers, with a percentage of 72.0±3.0% (FIG. 4c, sample #1), which is close to the predicted accuracy of the deep learning model (78.6%, FIG. 11). Similarly, it was correctly assigned polymer “AA0100AA” with an accuracy of 64.0±5.0% (sample #2), “AA00010AA” with an accuracy of 93.2±5.2% (sample #3), and “AA001000AA” with an accuracy of 94.5±4.2% (sample #4), which are consistent with the prediction from deep learning (i.e., 56.0%, 75.0%, and 90.0%, respectively). It was then measured a mixture sample in which polymers AA0000AA, AA0010AA, AA00100AA and AA00110AA were blindly mixed at equimolar ratio, recapitulating the composition with an accuracy of 14.0±2.0%, 20.0±4.0%, 20.0±4% and 16.0±4%, respectively (FIG. 4d), which is similar to the accuracy for 30 polymers classification given a equimolar concentration ratio of four polymers (i.e., 20.9%, 23.6%, 20.0%, 21.7%). Finally, a second mixture containing AA00010AA, AA00100AA and AA01000AA in an equimolar ratio was tested and an approximately equal assignment was observed (i.e., 29.0±1.0%, 27.0±3.0% and 21.0±1.0%), which is similar as the predicted accuracy (29.6%, 26.4% and 30.1%, FIG. 4e).

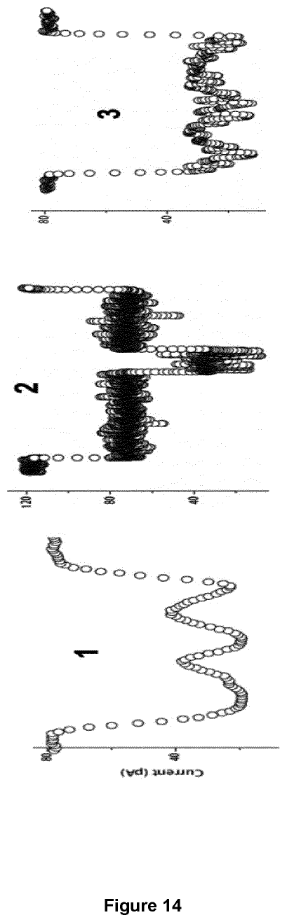

In an additional experiment, to show the potential of different type of “chemical bits”, single-channel recording setup and reading of polymers encoding single-bit information using an aerolysin pore were performed. As shown in FIG. 14 with magnifications of one single event, biomolecular data information media consisting of AA00100AA (1), AA00200AA (2) and AA00300AA (3) polymers (as described in FIG. 5) have been used and compared, showing the possibility of using different chemical structures for achieve the same data decoding purpose.

In conclusion, the inventors demonstrated that tailor-made informational polymers can be efficiently decoded by using, in the described exemplary embodiment, a variant of the aerolysin pore (K238A). In particular, the design of an optimal bio-inspired writing-reading framework allowed for single-bit resolution, which is unprecedented in analytical chemistry. The aerolysin pore structure can in principle be further tuned to optimize the translocation speed to allow efficient reading of longer polymers. On the other hand, the vast chemical space accessible to informational polymers can be further explored to enhance optimal decoding by biological nanopores. Importantly, informational polymers hybridized with DNA nucleobases keep some of the advantages of synthetic DNA used as support for data storage. For instance, different terminal nucleobases, which allow for more efficient capture by the nanopore, can be readily discriminated (FIG. 2) opening the possibility to use canonical DNA bases to define data structure in a format that can enable random access.

Writing-reading digital data using this biological-inspired nanopore-based platform can offer numerous advantages. First, single-bit resolution on the proposed informational polymer theoretically provides the opportunity to increase the information density of existing DNA-based solutions. Second, there is no need for amplification of detected molecules, lowering the time and cost of sample preparation and avoiding amplification errors. Furthermore, nanopore sensing does not require additional labelling and there is no theoretical upper limit for the reading length, further reducing the overall cost and workflow time. More importantly, nanopore sensing, which relies on an electrical readout, naturally enables large-scale parallelization based on already established technologies, allowing thus the construction of more affordable and portable devices for data management.

Mycobacterium Smegmatis Porin a (MspA) Nanopores

In an additional exemplary set-up, the inventors included the so-called M2-NNN mutant (D90N/D91N/D93N/D118R/E139K/D134R) of the MspA as a biological nanopore in a nanopore-based device.

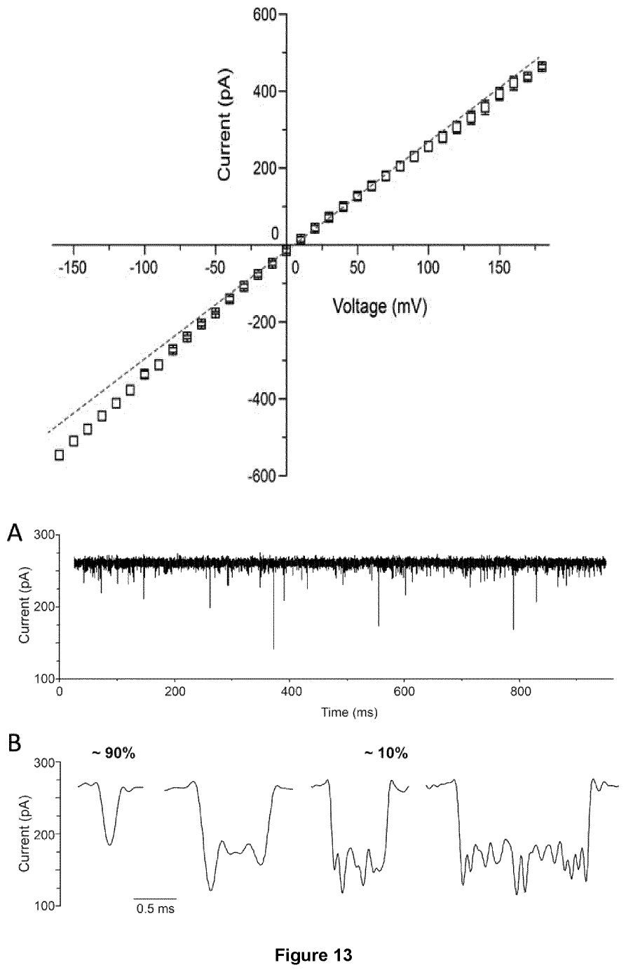

As shown in FIG. 13, unlike aerolysin, the number of signals collected by MspA is much less under the same polymer's concentration and set-up. In total, around −10% or the registered event perform as similar as observed with an aerolysin nanopore-based device and set-up, showing the possibility to use biological nanopores other than aerolysin for decoding purposes. Without being bound to any theory, it is considered that the difference in resolution may be due to the different sizes between the used nanopores (Aerolysin vs MspA).

Methods

Synthesis of the Macromolecular Analytes

The polymers used in the nanopore experiments were synthesized by automated phosphoramidite chemistry on an Expedite DNA synthesizer (Perseptive Biosystem 8900), as previously described (Al Ouahabi, A., et al, J. Am. Chem. Soc., 2015, April 8, doi: 10.1021/jacs.5b02639; Al Ouahabi, A., et al, ACS Macro Lett. 2015, September 10, doi: 10.1021/acsmacrolett.5b00606). All polymers were characterized by ESI-HRMS and their purity was controlled by anion-exchange HPLC, on an Agilent Apparatus equipped with a column Dionex BioLC DNAPac-PA100 and UV detectors (260 and 280 nm).

Aerolysin Productions

The aerolysin full length sequence was cloned in a pET22b vector with a C-terminal hexa-histidine tag to aid purification as described in Cao, C. et al., Nature Communications 2019, 10, 4918. The QuikChange II XL kit from Agilent Technologies was used for performing site-directed mutagenesis on the aerolysin gene, following manufacturer's instructions. The recombinant protein K238A was expressed and purified from BL21 DE3 pLys E. coli cells. Cells were grown to an optical density of 0.6-0.7 in Luria-Bertani (LB) media. Protein expression was induced by the addition of 1 mM isopropyl β-D-1-thiogalactopyranoside (IPTG) and subsequent growth over night at 20° C. Cell pellets were resuspended in lysis buffer (20 mM Sodium phosphate pH 7.4, 500 mM NaCl) mixed with cOmplete™ Protease Inhibitor Cocktail (Roche) and then lysed by sonication. The resulting suspensions were centrifuged (12.000 rpm for 35 min at 4° C.) and the supernatants were applied to an HisTrap HP column (GE Healthcare) previously equilibrated with lysis buffer. The protein was eluted with a gradient over 40 column volumes of elution buffer (20 mM Sodium phosphate pH 7.4, 500 mM NaCl, 500 mM Imidazole), and buffer exchanged into final buffer (20 mM Tris, pH 7.4, 500 mM NaCl) using a HiPrep Desalting column (GE Healthcare). The purified protein was flash frozen in liquid nitrogen and stored at −20° C.

Single-Channel Recording Experiments

Phospholipid of 1,2-Diphytanoyl-sn-glycero-3-phosphocholine powder (Avanti Polar Lipids, Inc., Alabaster, AL, USA) was dissolved in octane (Sigma-Aldrich Chemie GmbH, Buchs, Switzerland) for a final concentration of 1.0 mg per 100 μl. Purified K238A aerolysin mutant was diluted to the concentration of 0.2 μg/ml and then incubated with Trypsin-agarose (Sigma-Aldrich Chemie GmbH, Buchs, SG Switzerland) for 2 h under 4° C. temperature to activate the toxin for oligomerization. The solution was finally centrifuged to remove trypsin.

Nanopore single-channel recording experiments were performed by Orbit Mini equipment (Nanion, Munich, Germany). Phospholipid membranes were formed across a MECA 4 recording chip that contains a 2×2 array of circular microcavities in a highly inert polymer. Each cavity contains an individual integrated Ag/AgCl-microelectrode, and is able to record four artificial lipid bilayers in parallel. The current value leaps from 0.0 pA to nearly 80.0 pA once a single K238A aerolysin self-assembly into the membrane under the applied voltage of +100 mV. The measurement chamber temperature was set to 25 degree for all experiments.

Polymers in powder form were pre-diluted in water, to a stock concentration of 2.0 mg/ml and added to the cis side of the chamber in 1.0 M KCl solution buffered with 10 mM Tris and 1.0 mM EDTA (pH=7.4) to the final concentration of 20 μmol. All experiments shown here were repeated at least 10 different pores. The same conditions were used in experiments using the Mycobacterium smegmatis porin A (MspA) nanopores, such as the M2-NNN MspA mutant (D90N/D91N/D93N/D118R/E139K/D134R).

Current Signal Processing

The raw signals are segmented based on voltage discontinuities and large time-scale discontinuities in order to separate the signals segments where the pore is blocked or where a second pore is inserted into the membrane. For each segment, the open pore current distribution is measured by fitting a Gaussian function on the peak distribution of current with the highest mean current. The signals segments with an open pore current distribution of mean between 67 to 98 pA and standard deviation between 1.5 to 4.2 pA are kept.

The events are extracted using a current threshold at 3a from the open pore current distribution (FIG. 3a). The relative current Iᵣₑₗ=I/I₀ is computed from the mean open pore current (I₀). The cores of the events are extracted by removing the current drop at the beginning and end of the events using an adaptive current threshold. The dwell time, average relative current, current variation σᵣₑₗ=σ/σ₀ (σ₀ is the value of the open pore current standard deviation and a is the residual current standard deviation) and local extrema are computed. The events are selected based on the dwell time (0.4 to 30.0 ms) and the average relative current (15 to 60%) discarding the events which are too short or too long as well as removing the outliers. In average, this initial filtering procedure discard −10% of the events (FIG. 8).

In order to detect and label different level in the signal, the local relative current extrema are used to generate a Gaussian mixture model (GMM) with three components: low, high and transition level. The low and high Gaussian models correspond to the two main modes of the relative current extrema distribution. The transition level describe possible change of state between high and low level. Each event is segmented into low, high and transition levels of based on the level type with the highest probability predicted by the GMM. Finally, the transition levels which are not transition between high and low such as high-transition-high and low-transition-low are merged into a single high and low level respectively.

Finally to classify the current events, a machine learning approach was devised including two steps. The first one is the classification of every events and the second is the assessment of the quality of the prediction of the classifier (FIG. 3a). The neural network architecture for both the classification and the assessment is a long short-term memory (LSTM)32 neural network followed by a multilayer perceptron (MLP) using the position in time and relative current of the local extrema for each event as input features. The features are rescaled by a fixed factor to decrease the training time. The classifier is composed of a LSTM with state size 64 without any activation function followed by a 4 fully connected hidden layers of size 256 with hyperbolic tangent as activation functions and finally an output layer of size 30 with softmax activation function. The neural networks for the classification and assessment are trained together using a 3 parts loss functions. The first part is the full classification cross-entropy loss of the predictions from the classifier and the polymers label. The second part is the assessment cross-entropy loss between the predicted and actual prediction validity from the classifier. The third part is the reinforcement classification loss which is the full classification cross-entropy loss scaled by the assessment prediction.

While the invention has been disclosed with reference to certain preferred embodiments, numerous modifications, alterations, and changes to the described embodiments, and equivalents thereof, are possible without departing from the sphere and scope of the invention. Accordingly, it is intended that the invention not be limited to the described embodiments, and be given the broadest reasonable interpretation in accordance with the language of the appended claims.

### SEQUENCE LISTING

>sp|P09167|AERA_AERHY Aerolysin OS = Aeromonas
hydrophila GN = aerA (corresponds to region
24-493 of the wild type aerolysin
protein sequence)
SEQ ID NO: 1
AEPVYPDQLRLFSLGQGVCGDKYRPVNREEAQSVKSNIVG
MMGQWQISGLANGWWIMGPGYNGEIKPGTASNTWCYPTNP
VTGEIPTLSALDIPDGDEVDVQWRLVHDSANFIKPTSYLA
HYLGYAWVGGNHSQYVGEDMDVTRDGDGWVIRGNNDGGCD
GYRCGDKTAIKVSNFAYNLDPDSFKHGDVTQSDRQLVKTV
VGWAVNDSDTPQSGYDVTLRYDTATNWSKTNTYGLSEKVT
TKNKFKWPLVGETELSIEIAANQSWASQNGGSTTTSLSQS
VRPTVPARSKIPVKIELYKADISYPYEFKADVSYDLTLSG
FLRWGGNAWYTHPDNRPNWNHTFVIGPYKDKASSIRYQWD
KRYIPGEVKWWDWNWTIQQNGLSTMQNNLARVLRPVRAGI
TGDFSAESQFAGNIEIGAPVPLAADSKVRRARSVDGAGQG
LRLEIPLDAQELSGLGFNNVSLSVTPAANQ
>sp|P09167|AERA_AERHY Aerolysin OS = Aeromonas
hydrophila GN = aerA without C-terminal
propeptide (corresponds to region
24-445 of the wild type aerolysin
protein sequence)
SEQ ID NO: 2
AEPVYPDQLRLFSLGQGVCGDKYRPVNREEAQSVKSNIVG
MMGQWQISGLANGWIMGPGYNGEIKPGTASNTWCYPTNPV
TGEIPTLSALDIPDGDEVDVQWRLVHDSANFIKPTSYLAH
YLGYAWVGGNHSQYVGEDMDVTRDGDGWVIRGNNDGGCDG
YRCGDKTAIKVSNFAYNLDPDSFKHGDVTQSDRQLVKTVV
GWAVNDSDTPQSGYDVTLRYDTATNWSKTNTYGLSEKVTT
KNKFKWPLVGETELSIEIAANQSWASQNGGSTTTSLSQSV
RPTVPARSKIPVKIELYKADISYPYEFKADVSYDLTLSGF
LRWGGNAWYTHPDNRPNWNHTFVIGPYKDKASSIRYQWDK
RYIPGEVKWWDWNWTIQQNGLSTMQNNLARVLRPVRAGIT
GDFSAESQFAGNIEIGAPVPL

https://www.ncbi.nlm.nih.gov/protein/P09167.2

1. A molecular data storage medium comprising:
a) a header and a footer, each comprising or consisting of at least one unit of a first chemical species; and
b) a sequence-controlled polymeric chain of a second chemical species located between said header and said footer, said polymeric chain encoding for a desired bitstream-format media.

2. The molecular data storage medium of claim 1, wherein each monomer of the sequence-controlled polymeric chain encodes for one or more bits of said bitstream-format media.

3. The molecular data storage medium of claim 1, wherein said sequence-controlled polymeric chain is composed of a sequence of two or more types of monomers.

4. The molecular data storage medium of claim 1, wherein said unit of a first chemical species comprises a mononucleotide, a dinucleotide, an oligonucleotide or a polynucleotide.

5. The molecular data storage medium of claim 1, wherein said header and/or said footer comprise(s) a unit of a first chemical species having a sequence complementary to the unit of a first chemical species of a header and/or a footer of a second molecular data storage medium.

6. A method for encoding a bitstream-format information in a molecular data storage medium, the method comprising the steps of:
a) providing a desired media in a bitstream-format; and
b) converting said bitstream-format media into the molecular data storage medium of claim 1.

7. The method of claim 6, wherein the step of converting comprises synthesizing a sequence-controlled polymeric chain of a molecular data storage medium so that every monomer or group of monomers of said polymeric chain encodes for a bit or group of bits of said bitstream-format media.

8. A nanopore-based device for reading data stored in a molecular data storage medium, comprising:
a) a reservoir;
b) a membrane located on or within said reservoir in a way to split said reservoir in two facing chambers;
c) means for providing a voltage operatively connected to said reservoir; and
d) at least one nanopore spanning across the thickness of said membrane.

9. The nanopore-based device of claim 8, further comprising means for recording and analysing an electrical current.

10. The nanopore-based device of claim 8, wherein said membrane is a biological membrane.

11. The nanopore-based device of claim 8, wherein said at least one nanopore is a biological, macromolecular nanopore comprising a pore-forming toxins or a mutated pore-form toxins.

12. The nanopore-based device of claim 11, wherein said pore-forming toxin and/or mutated pore-form toxin is at least one of an aerolysin pore or a mutated aerolysin pore.

13. A method for decoding a bitstream-format information encoded in a molecular data storage medium, comprising the steps of:
a) providing a nanopore-based device for reading data stored in a molecular data storage medium according to, wherein the nanopore-based device comprises a reservoir; a membrane located on or within said reservoir in a way to split said reservoir in two facing chambers; means for providing a voltage operatively connected to said reservoir; and at least one nanopore spanning across the thickness of said membrane;
b) providing a molecular data storage medium according to claim 1 in a suitable buffer and in one chamber of the nanopore-based device reservoir;
c) providing a transmembrane voltage to said reservoir, thereby allowing the passage of said molecular data storage medium through said nanopore from one chamber to the opposed chamber of the reservoir; and
d) recording and analysing an electrical current during the passage of said molecular data storage medium through said nanopore, thereby decoding a bitstream-format information.

14. The method of claim 13, wherein the step of recording and analysing an electrical current comprises measuring a relative current distribution I/I₀, wherein I₀ is the value of the open nanopore current and I is the residual current value during the passage of said molecular data storage medium through said nanopore.

15. The molecular data storage medium of claim 3, wherein said unit of a first chemical species comprises a mononucleotide, a dinucleotide, an oligonucleotide or a polynucleotide.

16. The molecular data storage medium of claim 1, wherein said sequence-controlled polymeric chain of said second chemical species comprises or consists of at least one non-nucleic acid polymer chain.

17. The molecular data storage medium of claim 16, wherein said non-nucleic acid polymer chain is selected from the group consisting of amino acids, oligo- or polypeptides, synthetic monomers or polymers, and linear or branched carbohydrate chains.

18. The molecular data storage medium of claim 1, wherein said sequence-controlled polymeric chain of said second chemical species comprises or consists of a non-natural nucleic acid polymer chain.

19. The molecular data storage medium of claim 18, wherein said non-natural nucleic acid polymer chain comprises monomers selected from the following chemical structures: 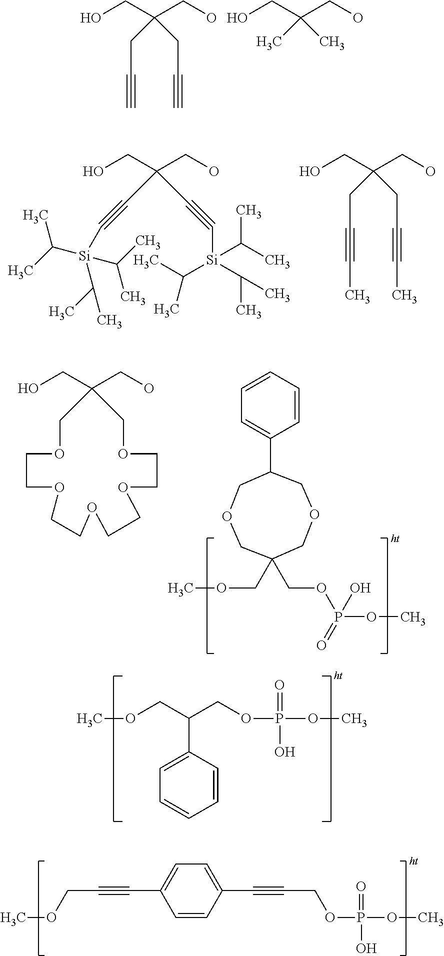 [chemical structure]
